(19) United States
(12) Patent Application Publication
(10) Pub. No.: US 2003/0149343 A1
(43) Pub. Date: Aug. 7, 2003

(54) Biometric based facility security

(75) Inventors: William G. Siegel, Wellington, FL (US); Greg L. Cannon, Boynton Beach, FL (US); Thomas Buss, Palm City, FL (US); George W. McClurg, Jensen Beach, FL (US); Mark Southwick, Lake Worth, FL (US); Richard Irving, Palm Beach Gardens, FL (US); Ron Kropp, West Palm Beach, FL (US); John Vandusen, Jupiter, FL (US); Trent Francis, Hobe Sound, FL (US)

Correspondence Address: STERNE, KESSLER, GOLDSTEIN & FOX PLLC, 1100 NEW YORK AVENUE, N.W., WASHINGTON, DC, 20005, US

(73) Assignee: Cross Match Technologies, Inc.

(21) Appl. No.: 10/255,112

(22) Filed: Sep. 26, 2002

Related U.S. Application Data
(60) Provisional application No. 60/324,517, filed on Sep. 26, 2001.

(51) Int. Cl. A61B 5/00

(52) U.S. Cl. 600/300

(57) ABSTRACT

Systems and methods are used to “check-in” and monitor individuals from an initial checkpoint of a facility to a final checkpoint of a facility. At the initial checkpoint, the systems and methods authenticate an identity of the individual, generate a biometric representation of the individual as an output, and associate the output to an object associated with the individual. The output can be machine-readable code, which can be encrypted or digitally signed. After this initial process, as the individual and the object associated with the individual travel through the facility, the individual's identity is re-authenticated one or more times either at discrete checkpoints or randomly throughout the facility. The object is also checked against the identity of the individual by using the output associate with the object to ensure the object should be with that individual.

### CROSS-REFERENCE TO RELATED APPLICATIONS

This application claims priority under 35 U.S.C. § 119(e) to U.S. Prov. App. No. 60/324,517 filed Sep. 26, 2001, which is incorporated by reference herein in its entirety.

The application is related to U.S. application Ser. No. 10/______, entitled “System And Method To Generate An Output Including A Machine Readable Code Representation Of Biometric Information” (Atty. Docket No. 1823.0480002), filed concurrently, which is incorporated by reference herein in its entirety.

### BACKGROUND OF THE INVENTION

1. Field of the Invention

The present invention relates to biometric detection and security.

2. Background Art

Access control systems are used to limit access to selected individuals. Some of these systems use biometric technologies to determine whether access for an individual will be granted or denied. A biometric is a unique, measurable characteristic or trait of a human being for automatically recognizing or verifying identity. For instance, fingerprint biometrics are largely regarded as an accurate method of biometric identification and verification. See, e.g., Roethenbaugh, G. Ed., Biometrics Explained (International Computer Security Association: Carlisle, Pa. 1998), pages 1-34, which is herein incorporated by reference in its entirety. Access control units (ACUs) may be placed locally or remotely to perform a biometric analysis on the individual, and determine whether access will be granted or denied.

Security at airports has becoming increasingly important. Conventional security techniques have included checking a passenger's photo identification, such as a driver's license or passport at ticket counter prior to issuing a boarding pass. A passenger is also required to pass through a security checkpoint prior to entering an airport concourse leading to boarding gates. The security checkpoint among other things includes a metal detector that passengers may walk through and an X-ray check of carry-on baggage. This does not provide any real security from people who are willing to fraudulently obtain identification with fake names and information to avoid being identified as who they really are.

What is needed is a biometric based airport security system and method that authenticates passengers and monitors movement of passengers with respect to movement of their belongings.

### BRIEF SUMMARY OF THE INVENTION

In one aspect of the present invention, a method is provided that includes (a) capturing biometric information from an individual at a location, (b) generating an output based on the biometric information, and (c) associating the output to an object that is associated with the individual. The method also includes (d) comparing the captured biometric information to stored biometric information, (e) generating a first comparison result signal based on the comparing step, and (f) transmitting the first comparison result signal to a predetermined location. The method further includes (g) capturing biometric information from the individual at another location, (h) comparing the captured biometric information from the another location to the stored biometric information or different stored biometric information, (i) generating a second comparison result signal based on the comparing step from the another location, and (j) transmitting the second comparison result signal to the predetermined location or different predetermined location. The method also includes (k) repeating steps (g)-(j) until the individual is at a final predetermined location.

In another aspect of the present invention, a system is provided that includes an initial checkpoint apparatus that is used to perform a first authentication process of an individual and a memory coupled to the initial check point apparatus that is used during the first authentication process of the individual. The system also includes an intermediate checkpoint apparatus that is used to perform a second authentication process of the individual and that is coupled to the memory. The system further includes a final checkpoint apparatus that is used to perform a final authentication process of the individual and that is coupled to the memory.

Further aspects, features, and advantages of the present inventions, as well as the structure and operation of the various embodiments of the present invention, are described in detail below with reference to the accompanying drawings.

### BRIEF DESCRIPTION OF THE DRAWINGS/FIGURES

The accompanying drawings, which are incorporated herein and form a part of the specification, illustrate the present invention and, together with the description, further serve to explain the principles of the invention and to enable a person skilled in the pertinent art to make and use the invention.

FIG. 1 is a block diagram showing an exemplary system for authenticating and monitoring an individual and an object associated with the individual, according to embodiments of the present invention.

FIG. 2 is a block diagram showing an exemplary initial checkpoint apparatus, according to embodiments of the present invention.

FIG. 3 is a block diagram showing an exemplary intermediate or final checkpoint apparatus, according to embodiments of the present invention.

FIG. 4 is a block diagram showing an exemplary object checking apparatus according, to embodiments of the present invention.

FIG. 5 is a block diagram showing an exemplary server in the system in FIG. 1, according to embodiments of the present invention.

FIG. 6 is a block diagram of an exemplary authentication portion in various apparatuses of the system in FIG. 1, according to embodiments of the present invention.

FIG. 7 is a flowchart depicting an authentication and monitoring method, according to embodiments of the present invention.

The present invention will now be described with reference to the accompanying drawings. In the drawings, like reference numbers indicate identical or functionally similar elements. Additionally, the left-most digit(s) of a reference number identifies the drawing in which the reference number first appears.

### DETAILED DESCRIPTION OF THE INVENTION

Overview

Embodiments of the present invention provide systems and methods to “check-in” and monitor individuals as they travel from an initial checkpoint of a facility to a final checkpoint of a facility. As an example, the systems and methods can be used to track passengers and their luggage and/or carry-on articles from a ticket counter, through a security checkpoint, to a boarding gate. Some advantages of this are: enhanced security with biometrics, flow control based on biometric verification and a sequence of checkpoints, checking of all passengers against criminal and/or terrorists databases, biometric information associated with the luggage and/or carry-on articles throughout the airport security environment, the additional biometric information associated with a passenger's article making curbside checking more secure, an ability to remotely check passengers and/or other users of the airport providing an additional level of security, and machine-readable code associated with checked luggage allowing a final check so that only when the passenger has cleared the boarding gate checkpoint will their luggage be loaded onto the plane.

At the initial checkpoint, present invention authenticates an identity of an individual, generates a biometric representation of the individual as an output, and associates the output to an object associated with the individual. The output can be machine-readable code, which can be encrypted or digitally signed. After this initial process, as the individual and the object associated with the individual travel through the facility, the individual's identity is re-authenticated one or more times either at discrete checkpoints or randomly throughout the facility. The object is also checked against the identity of the individual by using the output associated with the object to ensure the object should be with that individual.

The use of the term “biometric data” or “biometric information” throughout the specification can be representative of a biometric, a digital or other image of a biometric (e.g., a bitmap or other file), extracted digital or other information relating to the biometric (e.g., minutia of a fingerprint), etc.

A biometric as used throughout the specification may be a physical part of an individual, such as an eye, a finger, a limb, etc. An accessed system as used through the specification may be any known system that requires some limitation to entry, which can be an airport, a bus station, a mall, a school, a computer, electrical or mechanical equipment, a room, a hallway, a building, a section of a compound, etc. Matching used throughout the specification relates to matching either 1:1 to determine if the individual matches with whom he/she says he/she is, or 1:m, where m=all the enrollees, to determine if an individual is an enrollee at all.

Authentication and Monitoring System

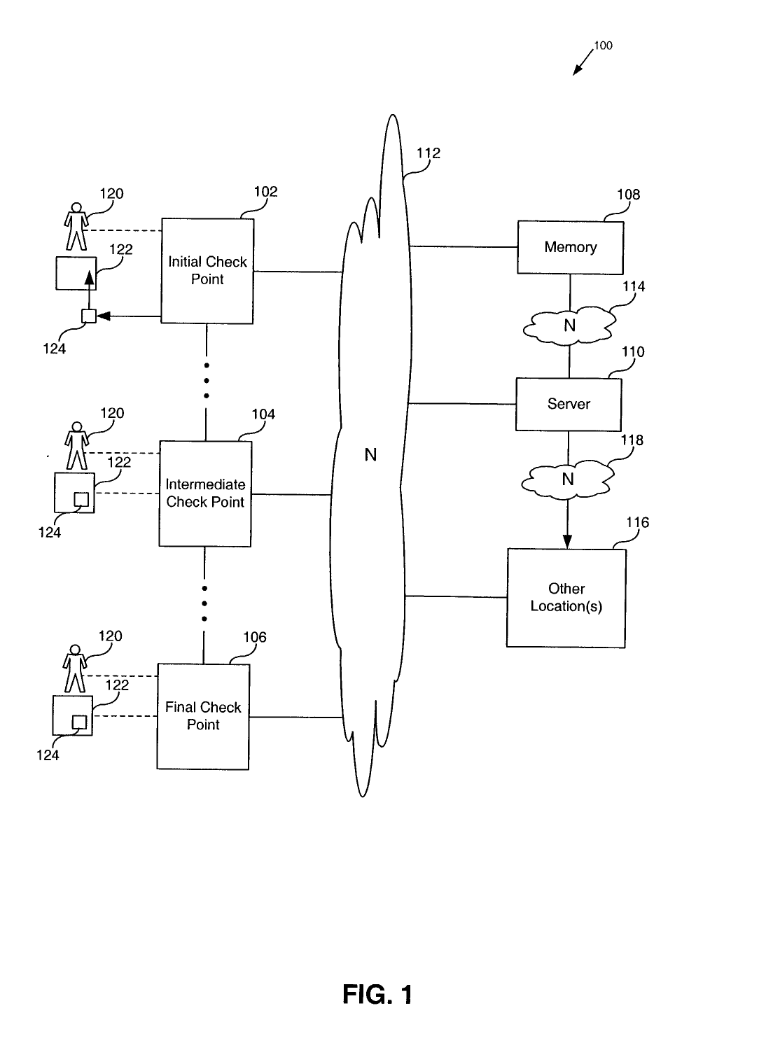

FIG. 1 is a block diagram showing an exemplary system 100 for authenticating and monitoring movement of individuals and objects associated with the individuals, according to embodiments of the present invention. In some embodiments, system 100 can be an accessed facility, such as an airport, that can track passengers and their carry-on and/or checked baggage. In other embodiments, system 100 can monitor movement of employees or personnel carrying badges or passes. System 100 includes an initial checkpoint apparatus 102, at least one intermediate checkpoint apparatus 104 (e.g., possibly 104a-n), and a final checkpoint apparatus 106. The checkpoint apparatus 102, 104, and 106 are coupled to a memory 108 either via a server 110 and/or via a network 112. Throughout the specification, use of “network” can mean an Intranet, an Internet, wired, wireless, or any other type of network than allows signals to be transmitted between devices and/or apparatus. Server 110 can be coupled to memory 108 via network 112 or another network 114. Server 110 can also be coupled to memory 108 via a cable, a bus, or other similar direct coupling. In some embodiments, server 110 can be coupled to another location 116, which can be similar to the server's location, via network 112 or another network 118. An individual 120 and an object 122 are authenticated at each checkpoint 102, 104, and 106, as will be described in more detail below. An output 124 from initial checkpoint 102 is associated with object 122, which will be described in more detail below.

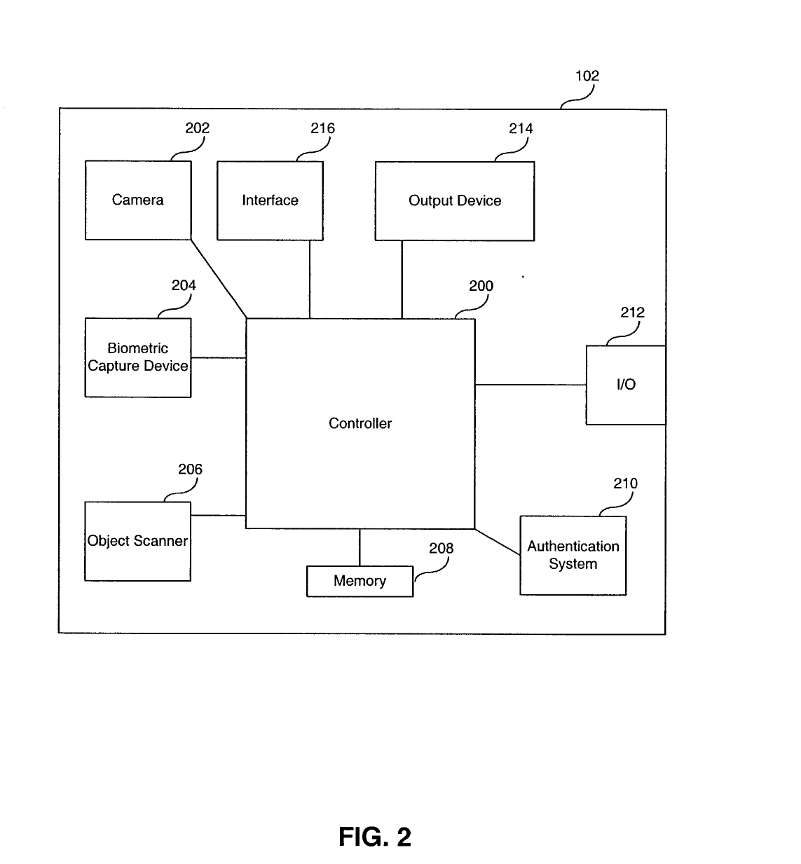

FIG. 2 is a block diagram showing an exemplary initial checkpoint apparatus 102 according to embodiments of the present invention. Central to initial checkpoint apparatus 102 is a controller 200, which can be coupled to and which can control all other elements in initial checkpoint apparatus 102. In various embodiments, controller 200 can control one or more of the following functions: image capture, minutia extraction, ticket scan optical recognition, data encryption, digital signature encoding, one (1-D) or two-dimensional (2-D) bar code encoding, and the like. In various embodiments initial checkpoint apparatus 102 can include: a camera 202 (e.g., a digital camera), a biometric capture device 204, an object scanner 206 (e.g., that scans a ticket), a memory 208, an authentication system 210, an input/output (I/O) device 212, an output device 214, and/or an interface 216 (e.g., a keyboard, keypad, touch screen, etc.). Details of the operation of initial checkpoint apparatus 102 and its various components will be described in more detail below.

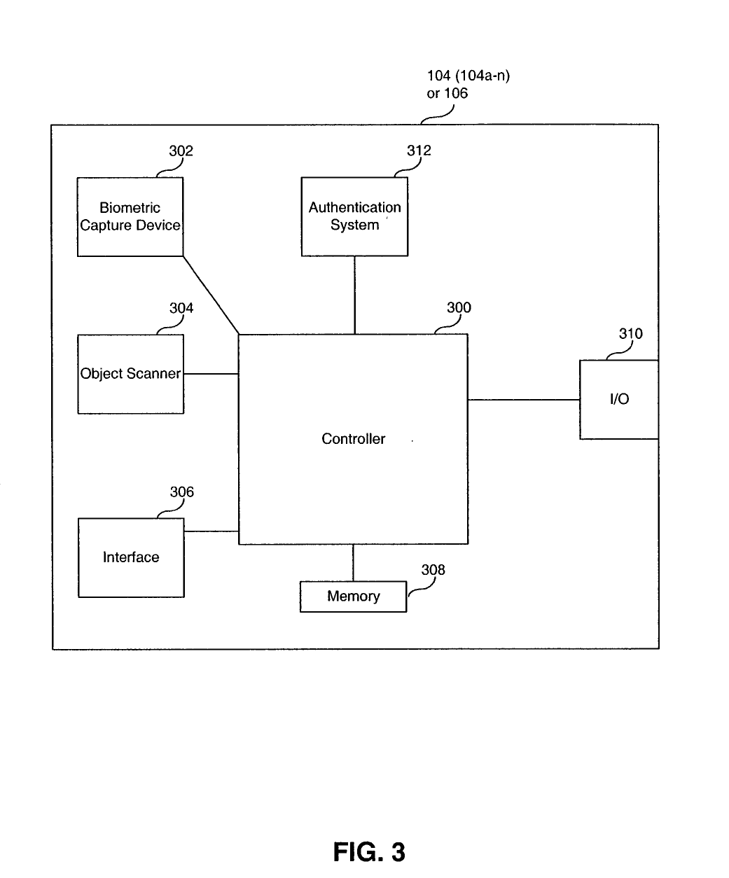

FIG. 3 is a block diagram showing an exemplary intermediate checkpoint apparatus 104 (or multiple intermediate checkpoint apparatus 104a-n) and final checkpoint apparatus 106 in system 100, according to embodiments of the present invention. Central to intermediate checkpoint apparatus 104 and/or final checkpoint apparatus 106 is a controller 300, which can be coupled to and which can control all other elements in intermediate checkpoint apparatus 104 and/or final checkpoint apparatus 106. Controller 300 can control similar functions controlled by controller 200. In various embodiments intermediate checkpoint apparatus 104 and/or final checkpoint apparatus 106 can include: a biometric capture device 302, an object scanner 304 (e.g., a machine-readable code reader, a bar code reader, a scanner, etc.), an interface 306 (e.g., a keyboard, keypad, touch screen, etc.), a memory 308, an I/O device 310, and/or an authentication system 312. Details of the operation of intermediate checkpoint apparatus 104 and final checkpoint apparatus 106 and their components will be described in more detail below.

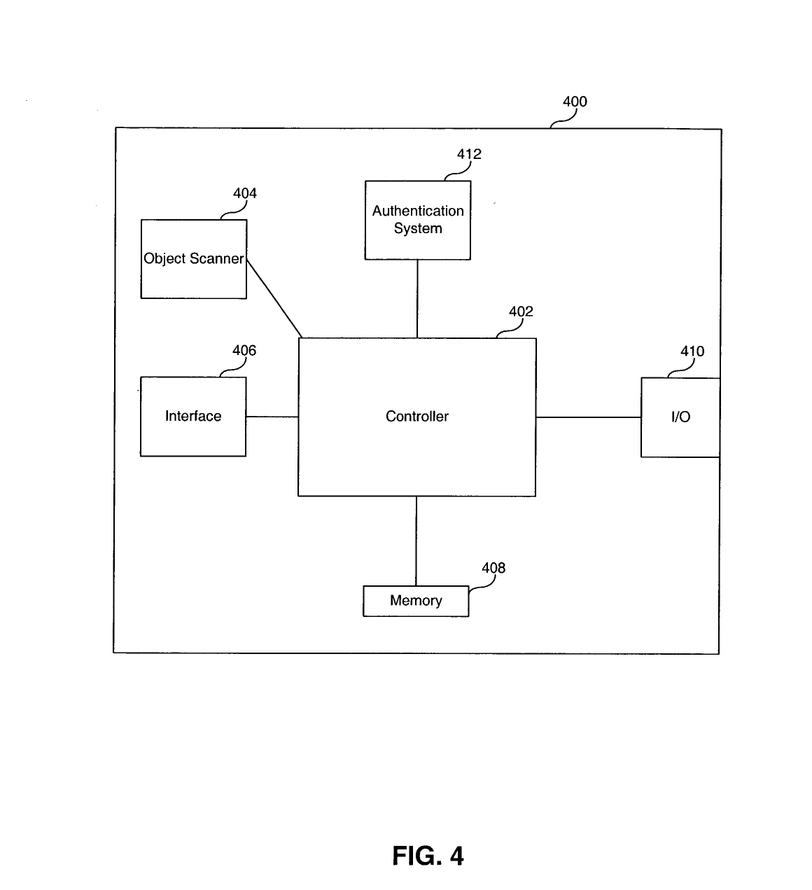

FIG. 4 is a block diagram showing an exemplary object checking apparatus 400 that is coupled (e.g., via a network, cable, bus, or other coupling) to system 100 according to embodiments of the present invention. Central to object checking apparatus 400 is a controller 402, which can be coupled to and which can control other elements in object checking apparatus 400. Object checking apparatus 400 includes one or more of the following elements: object scanner 404 (e.g., a machine-readable code reader, a bar code reader, a scanner, etc.), interface 406 (e.g., a keyboard, keypad, touch screen, etc.), memory 408, I/O device 410, and authentication system 412. Details of the operation of object checkpoint apparatus 400 and its components will be described in more detail below.

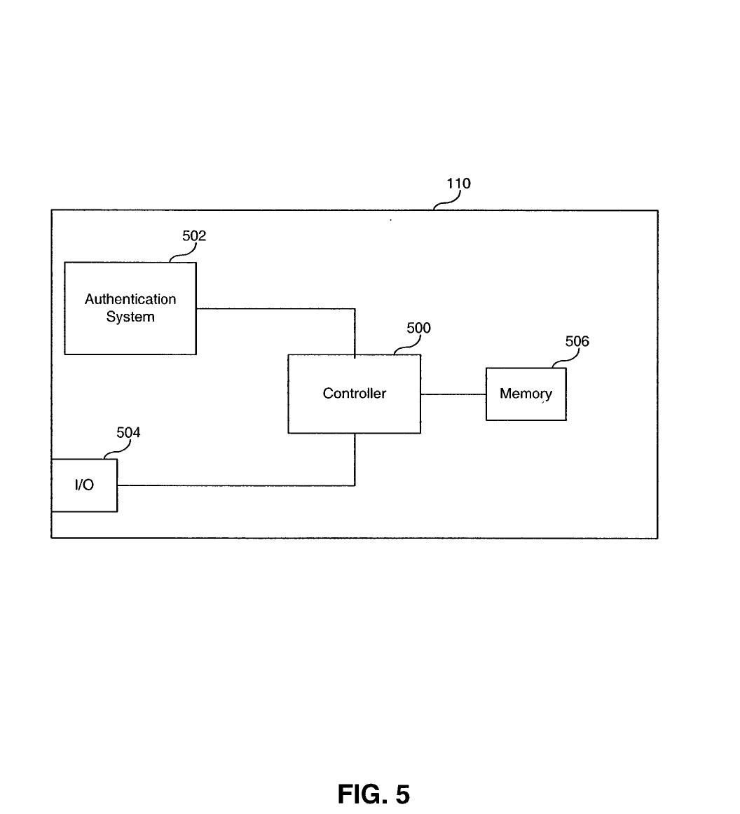

FIG. 5 is a block diagram showing an exemplary server 110 according to embodiments of the present invention. Central to server 110 is a controller 500, which can be coupled to an authentication system 502, an I/O device 504, and a memory 506.

It is to be appreciated that memories 208, 308, 408, and/or 506 can be local memory (e.g., RAM, ROM, etc.) used to store software, input data, processed data, or the like. In some embodiments, memory 108 stores previously captured or enrolled biometric data or information. Memory 108, for example, can store a database of biometric data or information (also referred to as a tracking database). In most embodiments, memories 208, 308, 408, and/or 506 do not store previously stored biometric data or information. In this way, biometric data is stored centrally, which improves security and make management and updating of the database of stored biometric data easier. Alternatively, memories 208, 308, 408, and/or 506 can store previously stored biometric data or information.

It is also to be appreciated that I/O devices 212, 310, 410, and/or 504 can be coupled via wires or wireless and via any known or future developed protocols to each other and all other components in system 100, as is known in the art.

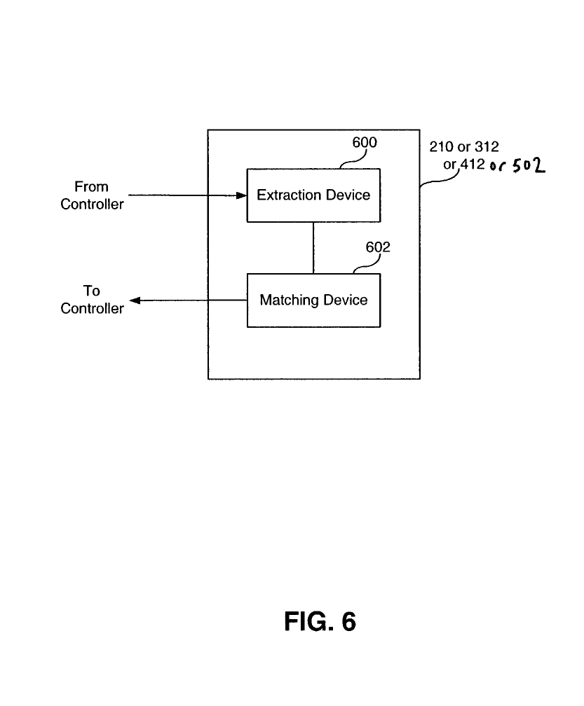

FIG. 6 is a block diagram showing exemplary authentication systems 210, 312, 412, and/or 502 according to embodiments of the present invention. Authentication systems 210, 312, 412, and/or 502 include an extraction device 600 and a matching device 602. Extraction device extracts biometric data (e.g., minutia) from biometric information (e.g., fingerprint information) captured by biometric capture device (e.g., a fingerprint scanner) 204 or 302, respectively. This extracted biometric data is matched (or compared) using matching device 602 to previously stored biometric data in memory 108. This is done to determine if the individual is one of a predetermined set of individuals whose biometric data is stored in memory 108. The predetermined set of individuals can correspond to terrorists, criminals, wanted fugitives, employees, vendors, etc. There is no limitation as to how many predetermined individuals can be pre-stored, or who they are. In other embodiments, authenticating systems 210, 312, and/or 412 can access biometric information from memory 108 and additional memory (not shown) at additional locations to provide a quick search memory and an expanded memory. Hence, biometric data associated with individuals that require immediate detection can be stored locally, while biometric data associated with individuals who do not need the immediate detection can be stored remotely.

Overall Method for Authentication and Monitoring Using the System

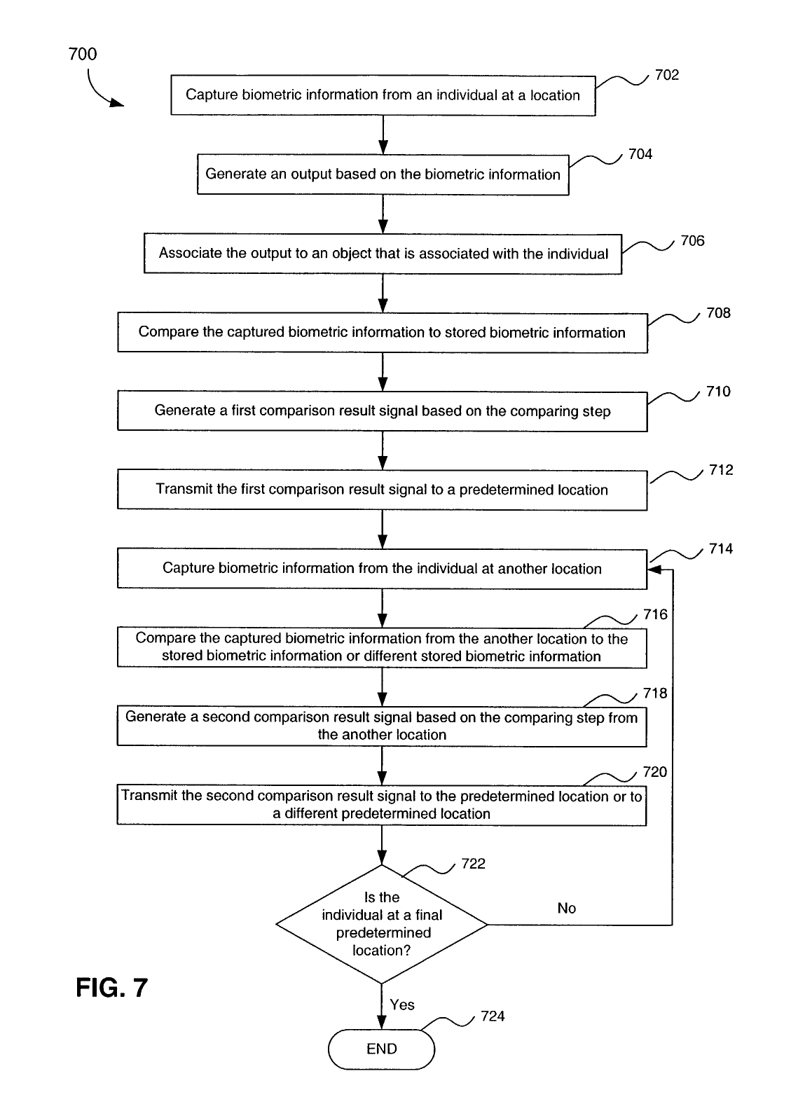

FIG. 7 is a flowchart depicting a method 700 according to embodiments of the present invention (steps 702-724). This method can be used to either identify random individuals (e.g., consumers or passengers) who are checking in at a location (e.g., an airport, bus station, port, customs, facility, etc.) and moving through that location, or to identify individuals in a preset group (e.g., employees, vendors, etc.) that are passing through the location (e.g., to go to their office or appointment).

During method 700, at step 702, biometric information (e.g., a fingerprint) from an individual at a location is captured. It is to be appreciated step 702 can be performed at a same time or on a same day as some of the below described subsequent steps are performed (e.g., for a consumer or a passenger), or on previous days or at previous times that some of the below described subsequent steps are performed (e.g., for an employee at hiring or a vendor at hiring).

At step 704, an output (e.g., a handheld device, a label, a voucher, any such device or other device with an adhesive material applied to—referred to as an adhesive device, or other object or device with a representation of the biometric information) is generated. An example of this process is described in more detail in U.S. application Ser. No. 10/______ entitled “System And Method To Generate An Output Including A Machine Readable Code Representation Of Biometric Information” (Atty. Docket No. 1823.0480002), filed concurrently, which is incorporated by reference herein in its entirety.

At step 706, the output is associated with an object (e.g., a carry-on bag, luggage, a boarding pass, a voucher, an employee badge or pass, a vendor or visitors badge or pass, etc.) that is associated with the individual.

At step 708, previously captured biometric information (either before or after extraction) is compared (or matched) to stored biometric information (or stored biometric data if after extraction).

At step 710, a first comparison result signal is generated based on the comparison.

At step 712, the first comparison result signal is transmitted to a predetermined location (e.g., where the captured biometric took place, locally to a public or private agency, remotely to a public or private agency, to subsequent locations the individual will encounter, etc.). During step 712, transactional information regarding a transaction that just occurred can also be sent.

Also during method 700, at step 714, biometric information from the individual is captured at another location (e.g., an intermediate checkpoint, a security checkpoint, randomly in the area using a wireless capture device, etc.).

At step 716, the captured biometric information is compared (or matched) to stored biometric information (or stored biometric data if after extraction). This stored biometric information (or data) can be the same as compared to in step 708 or different stored biometric information (or data).

At step 718, a second comparison signal is generated based on the comparing (or matching) step 716.

At step 720, the second comparison signal is transmitted to a predetermined location, which may be the same predetermined location as step 712 or a different predetermined location.

At step 722, a determination is made whether the individual is at a final predetermined location. If no, method 700 returns to step 714 and repeats steps 714 to 720. If yes, method 700 ends at step 724.

### EXAMPLE 1

Passenger Based Airport Security

In some embodiments, the present invention can provide biometric-based airport security. A biometric, such as a fingerprint, is detected at initial checkpoint apparatus 102 (e.g., at curbside or at a boarding pass station), intermediate checkpoint apparatus 104 (e.g., at a security checkpoint), and final checkpoint apparatus 106 (e.g., at a boarding gate). In the following example, these elements will be referred to as boarding pass station apparatus 102, security checkpoint apparatus 104, and boarding gate apparatus 106, for purposes of illustration, and not for purposes of limitation. Furthermore, individual 120 will be referred to as passenger 120, for illustrative purposes, and not for purposes of limitation. In various embodiments, a biometric capture device (e.g., a fingerprint scanner) 204 can be a VERIFIER fingerprint scanner, a MV5, a MV5+, or any other tabletop, wall-mounted, or hand-held fingerprint scanners, which are manufactured by Cross Match Technologies, Inc., a Florida corporation.

Using network 112, 114, and/or 118, a captured fingerprint can be checked against a local or remote memory (e.g., a database) 108 of stored fingerprint data. Thus, in this example, fingerprint data (e.g., a fingerprint image, extracted minutia information, etc.) is obtained from a finger of an individual (e.g., a passenger) that is presented at a boarding pass station apparatus 102 (e.g., at a ticket counter). The fingerprint data can then be compared using authentication system 210 or 502 with stored biometric data in memory 108. In various embodiments, memory 108 can provide for a simple check if it is a local database or a comprehensive background check of local, state, or federal law enforcement fingerprint data if it is a remote database. To expedite flow of passenger 120 through the airport, this check can be initiated at boarding pass section apparatus 102 when passenger 120 arrives, and can be performed and completed while passenger 120 proceeds to security checkpoint apparatus 104.

In addition, at the boarding pass station apparatus 102, an output (e.g., a label, sticker, etc.) 124 representative of detected fingerprint information (or other biometric data) can be printed out via output device 214 and applied to an object (e.g., a boarding pass, carry-on bags, luggage, a boarding pass, a badge, a pass, etc.) 122 associated with passenger 120. For illustrative purposes, output 124 will be referred to as label 124 in this example. For example, label 124 can be machine readable code (e.g., a one or two dimensional bar code) having data representative of the detected fingerprint data (e.g., the fingerprint image, extracted fingerprint minutia, etc.). Controller 200 can also encrypt, digitally sign, and/or encode the fingerprint data for additional security. Label 124 that includes the fingerprint data can be applied to a boarding pass object 122 and/or to any baggage (e.g., carry-on bags or checked bags) object 122.

At security checkpoint apparatus 104, passenger 120 can present both his or her finger and label(s) 124 on his or her boarding pass object 122 and/or carry-on bag objects 122. A biometric capture device (e.g., fingerprint scanner, such as the VERIFIER fingerprint scanner, or the like discussed above) 302 can then be used to scan a finger of passenger 120 to obtain fingerprint data (e.g., the fingerprint image, the extracted fingerprint minutia, etc). An object scanner (e.g., a bar code reader or scanner) 304 can then read the machine-readable code on label(s) 124. Authentication system 312 and controller (e.g., matcher and tracker module) can then check to determine whether the live fingerprint data obtained by fingerprint scanner 302 matches the fingerprint data obtained from labels 124. This is a one-to-one match that verifies the identity of the passenger 120 at intermediate checkpoint apparatus 104 as matching the passenger corresponding to label 124 printed by boarding pass station apparatus 102. In other embodiments, the check can be against fingerprint data stored in memory 108 or other memories.

During this check in process at security checkpoint apparatus 104, controller 300 can also check to determine whether passenger 120 has cleared the database check initiated at boarding pass station apparatus 102. Controller 300 can also check to make sure that the number of carry-on bag objects 122 with labels 124 scanned at security checkpoint apparatus 104 for passenger 120 matches the number of carry-on bag objects 122 identified and labeled for passenger 120 at check-in (either at curbside apparatus 102 or at ticket counter apparatus 102).

At boarding gate apparatus 106, passenger 120 again presents his or her finger to biometric capture device (e.g., a fingerprint scanner) 302 and presents corresponding labels 124 on his or her boarding pass object 122 and/or carry-on bag object(s) 122. Fingerprint scanner 302 then scans the finger to obtain fingerprint data (e.g., the fingerprint image, extracted fingerprint minutia, etc.). An object scanner 304 (e.g., a machine readable code scanner, a bar code reader, a scanner, or the like) then reads the bar code on each label 124. An authenticating system (e.g., a matcher and tracker module) 312 then checks to determine that the live fingerprint data obtained by fingerprint scanner 302 matches the fingerprint data obtained from labels 124. This is a one-to-one match that verifies the identity of passenger 120 at final checkpoint apparatus 106 as matching passenger 120 corresponding to label 124 printed by boarding pass station apparatus 102. In other embodiments, the check can be against fingerprint data stored in memory 108 or other memories.

During this process, controller 300 can also check to determine whether passenger 120 has cleared the database check initiated at boarding pass station apparatus 102. Controller 300 can also check to make sure that the number of carry-on bag objects 122 with labels 124 scanned at boarding gate apparatus 102 for passenger 120 matches the number of carry-on bag objects 122 identified and labeled for passenger 120 at check-in (either at curbside apparatus 102 or at ticket counter apparatus 102). Controller 300 can also check database information tracking the flow of passenger 120 from end-to-end through the system 100 (e.g., the airport). Thus, controller 300 can check tracking data to ensure that passenger 120 has proceeded through earlier stages from boarding pass station apparatus 102, through security checkpoint apparatus 104 to boarding gate apparatus 106.

In other embodiments, checked bag objects 122 or luggage objects 122 having labels 124 with fingerprint data are scanned by object checking apparatus 400 before or during loading of the luggage objects 122 onto a plane (not shown). Tracking data generated during this check can be stored for the bag objects 122 in memory (e.g., a database) 408. When passenger 120 is at boarding gate apparatus 106, a check can then also be made by controller 300 to verify data corresponding to the actual number of bag objects 122 checked by passenger 120 and loaded on the plane matches data corresponding to a number of bag objects 122 checked by passenger 120 at initial checkpoint apparatus 102. Also, a check can be made to verify that passenger 120 who checked bag objects 122 at initial checkpoint apparatus 102 has actually boarded the plane. This will ensure that luggage objects 122 for passengers 120 not boarding the plane is either not placed on the plane or removed from the plane.

### EXAMPLE 2

Employee or Personnel Based Airport Security

In other embodiments, the present invention can also function as an airport pass or badge checking system and method. In these embodiments, initial checkpoint apparatus 102 is used for enrollment purposes. Intermediate checkpoint apparatus 104 (e.g., a mobile airport pass checking apparatus) can be used to check an employee individual 120 or other personnel with passes/badges objects 122 having labels 124. It is to be appreciated, this embodiment can also involved checking passenger's 120 with passes/badges objects 122 having labels 124. In one embodiment, mobile airport pass checking apparatus 104 is a small computer device including object scanner (e.g., bar code scanner) 304, user interface 306, a controller (e.g., a processor) 300, and an I/0 device 310 (e.g., a wireless interface, such as an 802.11b interface). A biometric capture device (e.g., a fingerprint scanner, including but not limited to, a MV5 or MV5+ hand-held fingerprint scanner available from Cross Match Technologies, Inc., a Florida corporation) 302 can be used to scan a finger to obtain biometric (e.g., fingerprint) information or data (e.g., fingerprint image, extracted fingerprint minutia, etc) and forward it for checking at a local or remote memory (e.g., database) 108 and for tracking location, status or other information about the detected individual 120.

Other Examples Uses of the Authentication and Monitoring System

In still other embodiments, camera (e.g. digital camera) 202 can capture an image of a face or retina of individual 120 as other biometrics. Then, the image information can be either extracted and matched to stored image information in memory 108 or matched without extractions using authentication system 210. Note that this can be done secretly, i.e., without an individual's knowledge, for various reasons.

Conclusion

While various embodiments of the present invention have been described above, it should be understood that they have been presented by way of example only, and not limitation. It will be apparent to persons skilled in the relevant art that various changes in form and detail can be made therein without departing from the spirit and scope of the invention. Thus, the breadth and scope of the present invention should not be limited by any of the above-described exemplary embodiments, but should be defined only in accordance with the following claims and their equivalents.

What is claimed is:

1. A method comprising the steps of:
(a) capturing biometric information from an individual at a location;
(b) generating an output based on the biometric information;
(c) associating the output to an object that is associated with the individual;
(d) comparing the captured biometric information to stored biometric information;
(e) generating a first comparison result signal based on said comparing step;
(f) transmitting the first comparison result signal to a predetermined location;
(g) capturing biometric information from the individual at another location;
(h) comparing the captured biometric information from the another location to the stored biometric information or different stored biometric information;
(i) generating a second comparison result signal based on said comparing step from the another location;
(j) transmitting the second comparison result signal to the predetermined location or different predetermined location; and
(k) repeating steps (g)-(j) until the individual is at a final predetermined location.

2. The method of claim 1, further comprising the step of designating said location and said predetermined location as being a same location.

3. The method of claim 2, wherein the same location is an airport.

4. The method of claim 1, further comprising the step of designating said predetermined location and the different predetermined location to be a remote location.

5. The method of claim 1, further comprising the step of designating the predetermined location and the different predetermined location to be a governmental agency.

6. The method of claim 1, wherein said generating step (b) generates an adhesive device with a machine-readable code as the output.

7. The method of claim 6, wherein said generating step (b) generates a label as the adhesive device.

8. The method of claim 1, wherein said generating step (b) generates a machine-readable code as the output.

9. The method of claim 8, wherein said generating step (b) includes a step of forming a representation of the biometric information as the machine-readable code.

10. The method of claim 1, wherein the object comprises a handheld device, and wherein said associating step (c) includes associating the output to a section of the handheld device.

11. The method of claim 10, wherein said associating step (c) includes a step of affixing the output on the section of the handheld device.

12. The method of claim 11, wherein the handheld device is a ticket.

13. The method of claim 11, wherein the handheld device is a voucher.

14. The method of claim 11, wherein the handheld device is a badge.

15. The method of claim 11, wherein the handheld device is a pass.

16. The method of claim 11, wherein said affixing step includes printing a machine-readable code directly on the section of the object.

17. The method of claim 11, further comprising the step of printing a machine-readable code on an adhesive device, wherein the adhesive device is coupled to the section of the object during said affixing step.

18. The method of claim 1, further comprising the step of establishing a initial check point in an airport as the location.

19. The method of claim 1, further comprising the step of establishing a specific location as the another location.

20. The method of claim 1, further comprising the step of using a random location as the another location.

21. The method of claim 20, further comprising secretly performing said capturing step at the another location.

22. The method of claim 20, wherein said capturing step (g) is performed with a handheld capture device.

23. The method of claim 1, further comprising the step of establishing an intermediate checkpoint in an airport as the another location.

24. The method of claim 1, further comprising the step of establishing a security checkpoint in an airport as the another location.

25. The method of claim 1, further comprising establishing the final predetermined location to be a gate at an airport.

26. The method of claim 1, further comprising the steps of:
capturing information from the output at the another location; and
verifying the object belongs to the individual.

27. The method of claim 1, further comprising the step of:
verifying the individual has been authenticated at the final predetermined location before allowing a remotely located one of the object to pass the final predetermined location.

28. The method of claim 27, wherein:
said final predetermined location is a gate at an airport; and
the remotely located object is luggage being transported from the airport to a plane.

29. The method of claim 1, further comprising the steps of:
storing the stored biometric information locally relative to the location; and
storing the different stored biometric information locally relative to the another location.

30. The method of claim 29, wherein the stored biometric information and the different stored biometric information are a predetermined list of most wanted individuals as determined by governmental agencies.

31. The method of claim 30, wherein the most wanted individuals are most wanted terrorists.

32. The method of claim 30, wherein the most wanted individuals are most wanted criminals.

33. The method of claim 1, further comprising the steps of:
storing the stored biometric information remotely relative to the location; and
storing the different stored biometric information remotely relative to the another location.

34. The method of claim 1, further comprising the steps of:
storing the stored biometric information remotely relative to the location; and
storing the different stored biometric information locally relative to the another location.

35. The method of claim 1, further comprising the step of:
capturing transactional information after said capturing step (a); and
transmitting the transaction information to the predetermined location.

36. The method of claim 35, wherein the transactional information is at least one of date, time, location, identity, and details of services a transaction; details of products of a transaction.

37. A system comprising:
an initial checkpoint apparatus that is used to perform a first authentication process of an individual;
a memory coupled to said initial check point apparatus that is used during the first authentication process of the individual;
an intermediate checkpoint apparatus that is used to perform a second authentication process of the individual and that is coupled to said memory; and
a final checkpoint apparatus that is used to perform a final authentication process of the individual and that is coupled to said memory.

38. The system of claim 37, further comprising a network, wherein said initial checkpoint apparatus, said intermediate checkpoint apparatus, and said final checkpoint apparatus are coupled to said memory via said network.

39. The system of claim 38, wherein said network is a Intranet.

40. The system of claim 38, wherein said network is an Internet.

41. The system of claim 38, wherein said network is a wired network.

42. The system of claim 38, wherein said network is a wireless network.

43. The system of claim 37, wherein said initial checkpoint apparatus comprises:
a biometric capture device that captures biometric information from the individual;
an authentication portion coupled to the biometric capture device that generates biometric data from the biometric information and that compares the biometric data to biometric data stored in said memory; and
an output device that is used to generate an machine readable code based on said biometric information or said biometric data, where said machine readable code is associated with an object associated with the individual.

44. The system of claim 43, wherein the initial checkpoint apparatus further comprises a second biometric capture device.

45. The system of claim 44, wherein:
said biometric capture device is a fingerprint scanner; and
said second biometric capture device is a digital camera.

46. The system of claim 43, wherein said machine-readable code is printed on a label with an adhesive portion.

47. The system of claim 43, wherein said machine-readable code is printed on a section of said object.

48. The system of claim 43, wherein said machine-readable code is a bar code.

49. The system of claim 43, wherein said machine-readable code is a two-dimensional bar code.

50. The system of claim 43, wherein said machine-readable code is digitally signed.

51. The system of claim 43, wherein said machine-readable code is encrypted.

52. The system of claim 43, wherein said authentication portion comprises:
an extracting device that extracts the biometric data from the captured biometric information; and
a matching device that compares the biometric data to the stored data.

53. The system of claim 43, wherein said initial checkpoint apparatus further comprises a ticket scanner that scans a ticket of the individual.

54. The system of claim 43, wherein said object is a boarding pass.

55. The system of claim 43, wherein said object is a tag for a carry-on article.

56. The system of claim 43, wherein said object is a tag for stow away luggage.

57. The system of claim 37, wherein said intermediate checkpoint apparatus is positioned randomly throughout a designated area.

58. The system of claim 37, wherein said intermediate checkpoint apparatus is a handheld authentication device.

59. The system of claim 37, wherein said intermediate checkpoint apparatus is positioned at a security checkpoint in an airport.

60. The system of claim 37, wherein said intermediate checkpoint apparatus comprises:
a biometric capture device that captures biometric information from the individual;
an authentication portion coupled to the biometric capture device that generates biometric data from the biometric information and that compares the biometric data to biometric data stored in said memory; and
a reading device that reads said machine-readable code.

61. The system of claim 60, wherein said authentication portion comprises:
an extracting device that extracts the biometric data from the captured biometric information; and
a matching device that compares the biometric data to the stored data.

62. The system of claim 37, wherein said intermediate checkpoint apparatus comprises:
a biometric capture device that captures biometric information from the individual;
an authentication portion coupled to the biometric capture device that generates biometric data from the biometric information and that compares the biometric data to biometric data stored in said memory; and
a reading device that reads said machine-readable code.

63. The system of claim 62, wherein said authentication portion comprises:
an extracting device that extracts the biometric data from the captured biometric information; and
a matching device that compares the biometric data to the stored data.

64. The system of claim 37, wherein said memory is positioned locally with respect to said initial, intermediate, and final checkpoint apparatus and said memory stores biometric data for a predetermined number of individuals.

65. The system of claim 64, wherein said predetermined number of individuals are terrorists.

66. The system of claim 64, wherein said predetermined number of individuals are criminals.

67. The system of claim 37, wherein said memory is positioned remotely with respect to said initial, intermediate, and final checkpoint apparatus and said memory stores biometric data for a predetermined number of individuals.

68. The system of claim 67, wherein said predetermined number of individuals are terrorists.

69. The system of claim 67, wherein said predetermined number of individuals are criminals.

70. The system of claim 67, wherein said initial, intermediate, and final checkpoints are located in an airport.

71. The system of claim 43, further including an object checking apparatus that determines a position of the object with respect to the individual.

72. The method of claim 1, further comprising the steps of determining a position of the object with respect to the individual at the another location and the final predetermined location.

73. A biometric-based airport security system comprising:
a ticket counter apparatus that is used to perform a first authentication process of a passenger;
a memory coupled to said ticket counter apparatus that is used during the first authentication process of the passenger;
an security checkpoint apparatus that is used to perform a second authentication process of the passenger and that is coupled to said memory; and
a boarding gate apparatus that is used to perform a final authentication process of the passenger and that is coupled to said memory.

74. The system of claim 73, further comprising a network, wherein said ticket counter apparatus, said security checkpoint apparatus, and said boarding gate apparatus are coupled to said memory via said network.

75. The system of claim 74, wherein said network is a Intranet.

76. The system of claim 74, wherein said network is an Internet.

77. The system of claim 74, wherein said network is a wired network.

78. The system of claim 74, wherein said network is a wireless network.

79. The system of claim 73, wherein said ticket counter apparatus comprises:
a biometric capture device that captures biometric information from the passenger;
an authentication portion coupled to the biometric capture device that generates biometric data from the biometric information and that compares the biometric data to biometric data stored in said memory; and
an output device that is used to generate an machine readable code based on said biometric information or said biometric data, where said machine readable code is associated with an object associated with the passenger.

80. The system of claim 79, wherein the ticket counter apparatus further comprises a second biometric capture device.

81. The system of claim 80, wherein:
said biometric capture device is a fingerprint scanner; and
said second biometric capture device is a digital camera.

82. The system of claim 79, wherein said machine-readable code is printed on a label with an adhesive portion.

83. The system of claim 79, wherein said machine-readable code is printed on a section of said object.

84. The system of claim 79, wherein said machine-readable code is a bar code.

85. The system of claim 79, wherein said machine-readable code is a two-dimensional bar code.

86. The system of claim 79, wherein said machine-readable code is digitally signed.

87. The system of claim 79, wherein said machine-readable code is encrypted.

88. The system of claim 79, wherein said authentication portion comprises:
an extracting device that extracts the biometric data from the captured biometric information; and
a matching device that compares the biometric data to the stored data.

89. The system of claim 79, wherein said ticket counter apparatus further comprises a ticket scanner that scans a ticket of the passenger.

90. The system of claim 79, wherein said object is a boarding pass.

91. The system of claim 79, wherein said object is a tag for a carry-on article.

92. The system of claim 79, wherein said object is a tag for stow away luggage.

93. The system of claim 73, wherein said security checkpoint apparatus is positioned randomly throughout a designated area.

94. The system of claim 73, wherein said security checkpoint apparatus is a handheld authentication device.

95. The system of claim 73, wherein said security checkpoint apparatus comprises:
a biometric capture device that captures biometric information from the passenger;
an authentication portion coupled to the biometric capture device that generates biometric data from the biometric information and that compares the biometric data to biometric data stored in said memory; and
a reading device that reads said machine-readable code.

96. The system of claim 95, wherein said authentication portion comprises:
an extracting device that extracts the biometric data from the captured biometric information; and
a matching device that compares the biometric data to the stored data.

97. The system of claim 73, wherein said security checkpoint apparatus comprises:
a biometric capture device that captures biometric information from the passenger;
an authentication portion coupled to the biometric capture device that generates biometric data from the biometric information and that compares the biometric data to biometric data stored in said memory; and
a reading device that reads said machine-readable code.

98. The system of claim 97, wherein said authentication portion comprises:
an extracting device that extracts the biometric data from the captured biometric information; and
a matching device that compares the biometric data to the stored data.

99. The system of claim 73, wherein said memory is positioned locally with respect to said ticket counter, security checkpoint, and boarding gate apparatus and said memory stores biometric data for a predetermined number of passengers.

100. The system of claim 99, wherein said predetermined number of passengers are terrorists.

101. The system of claim 99, wherein said predetermined number of passengers are criminals.

102. The system of claim 73, wherein said memory is positioned remotely with respect to said ticket counter, security checkpoint, and boarding gate apparatus and said memory stores biometric data for a predetermined number of passengers.

103. The system of claim 102, wherein said predetermined number of passengers are terrorists.

104. The system of claim 102, wherein said predetermined number of passengers are criminals.

105. The system of claim 79, further including an object checking apparatus that determines a position of the object with respect to the individual.
